(19) United States
(12) Patent Application Publication
(10) Pub. No.: US 2019/0131013 A1
(43) Pub. Date: May 2, 2019

(54) PHARMACEUTICAL WASTE SORTING ASSISTANCE SYSTEM AND METHOD THEREOF

(71) Applicants: Mukul KELKAR, West Sacramento, CA (US); Duone JACKSON, West Sacramento, CA (US)

(72) Inventors: Mukul KELKAR, West Sacramento, CA (US); Duone JACKSON, West Sacramento, CA (US)

(21) Appl. No.: 16/174,069

(22) Filed: Oct. 29, 2018

Related U.S. Application Data
(60) Provisional application No. 62/578,524, filed on Oct. 30, 2017.

(51) Int. Cl. G16H 40/20; G16H 70/40; G06Q 10/00

(52) U.S. Cl. CPC G16H 40/20; G06K 2019/06253; G06Q 10/30; G16H 70/40

(57) ABSTRACT

A sorting assistance system and method for sorting of pharmaceutical wastes is disclosed. The system for assisting in sorting of pharmaceutical wastes may include a server in communication with at least one database storing composition data of a plurality of pharmaceuticals, and an assistance device in communication with a scanner device and the database. The assistance device (a) reads a machine readable code affixed to a pharmaceutical using the scanner device, (b) identifies the pharmaceutical upon reading the machine readable code, (c) receives the composition data associated with the identified pharmaceutical from the at least one database, (d) determines a category of the pharmaceutical using the composition data and a logic engine, (e) determines a bin out of the plurality of bins for placing the pharmaceutical based on the identified category, and (f) displays the visual indicator corresponding to the determined bin, on a screen of the assistance device.

### TECHNICAL FIELD

The present disclosure generally relates to waste segregation systems and more particularly to a pharmaceutical waste sorting assistance system and method thereof for sorting or segregating pharmaceutical wastes in hospitals, pharmacies and medical facilities.

### BACKGROUND

The waste management industry has multiple facets and categories of wastes that are removed from various premises. The hospitals and the medical facilities waste industry is strictly regulated by Environmental Protection Agency (EPA) and Food and Drug Administration (FDA) to ensure that the risk of diseases are kept away from humans, pets, other animals and hence, the environment. A few of the categories of wastes that are hauled from a hospital are regulated medical waste (RMW), sharps, solid waste, recyclable waste and pharmacy (pharma) waste.

Typically, the pharmacy waste is any waste which contains medicinal drugs that are unused, partially used, expired, contaminated drugs, contaminated remaining containers or those no longer needed. In particularly, the pharmacy waste alone may be categorised into sub-categories such as non-hazardous, hazardous, hazardous-P/U (P-listed and U-listed substances), flammable or toxic, for example. The hazardous and toxic wastes generally have the potential to cause or significantly contribute to an increase in mortality or an increase in serious reversible or irreversible illness. Hence, such wastes pose a substantial hazard to human health or the environment when improperly treated, stored, transported, disposed of, or otherwise managed. The process of collection and removal of all such categories of waste has been established for many years and are quite standardized to ensure consistency, safety and reliability.

The pharma medical waste is of particular importance to both the hospitals, pharmacies and haulers due to its high cost of disposal as well as concern of being used illegally for recreational and other purposes. It is also important to ensure that hazardous pharma wastes do not end up in non-hazardous containers. Very high fines may be implemented by both the FDA and EPA to ensure such cross-contamination does not exist. In the case where non-hazardous materials are mixed with hazardous materials, then the disposal cost can significantly rise. A simple, reliable and scalable technological solution to implement compliance and yet to prevent rising costs of pharma waste disposal has been absent for the medical waste industry.

### SUMMARY OF THE DISCLOSURE

Thus, there exists a need for a system and method which mitigates some of the disadvantages of the state of the art.

This summary is provided to introduce a selection of concepts in a simple manner that are further described in the detailed description of the disclosure. This summary is not intended to identify key or essential inventive concepts of the subject matter nor is it intended for determining the scope of the disclosure.

A sorting assistance system and method for sorting of pharmaceutical wastes is disclosed. In some embodiments of the present disclosure, the sorting assistance system for assisting in sorting of pharmaceutical wastes into a plurality of bins comprises, a server in communication with at least one database storing composition data of a plurality of pharmaceuticals, and an assistance device in communication with a scanner device and the at least one database, wherein the assistance device is configured for, (a) reading a machine readable code affixed to a pharmaceutical to be sorted using the scanner device, the machine readable code comprising a pharmaceutical identifier, (b) identifying the pharmaceutical upon reading the machine readable code, (c) receiving the composition data associated with the identified pharmaceutical from the at least one database, (d) determining a category of the pharmaceutical using the composition data and a logic engine, the logic engine defining one or more rules for determining the category, (e) determining a bin out of the plurality of bins for placing the pharmaceutical based on the identified category, wherein each bin corresponds to one of the category and is identified with an unique visual indicator, and (f) displaying the visual indicator corresponding to the determined bin, on a screen of the assistance device.

### BRIEF DESCRIPTION OF THE FIGURES

The disclosure will be described and explained with additional specificity and detail with the accompanying figures in which:

FIG. 1 illustrates an exemplary representation of a sorting assistance system for assisting in sorting of pharmaceutical wastes in accordance with an embodiment of the present disclosure; and

FIG. 2 illustrates a pharmaceutical waste sorting method using sorting assistance system in accordance with an embodiment of the present disclosure.

Further, persons skilled in the art to which this disclosure belongs will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those of ordinary skill in the art having benefit of the description herein.

### DETAILED DESCRIPTION OF THE EXEMPLARY EMBODIMENTS

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications to the disclosure, and such further applications of the principles of the disclosure as described herein being contemplated as would normally occur to one skilled in the art to which the disclosure relates are deemed to be a part of this disclosure.

It will be understood by those skilled in the art that the foregoing general description and the following detailed description are exemplary and explanatory of the disclosure and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or a method. Similarly, one or more devices or sub-systems or elements or structures or components preceded by “comprises . . . a” does not, without more constraints, preclude the existence of other devices, other sub-systems, other elements, other structures, other components, additional devices, additional sub-systems, additional elements, additional structures, or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail with reference to the accompanying figures.

The embodiments herein disclose sorting assistance system for assisting in sorting pharmaceutical wastes into a plurality of bins. In some embodiments, the sorting assistance system comprises a server in communication with at least one database, the database storing composition data of a plurality of pharmaceuticals, and one or more assistance device in communication with the server, wherein the one or more assistance devices are located at various medical facilities for assisting users in sorting of pharmaceutical wastes generated at the medical facilities. The medical facilities as described herein may include hospitals, clinics, outpatient care centres, specialized care centres, pharmacies, pharmaceutical storage and distribution centres, for example.

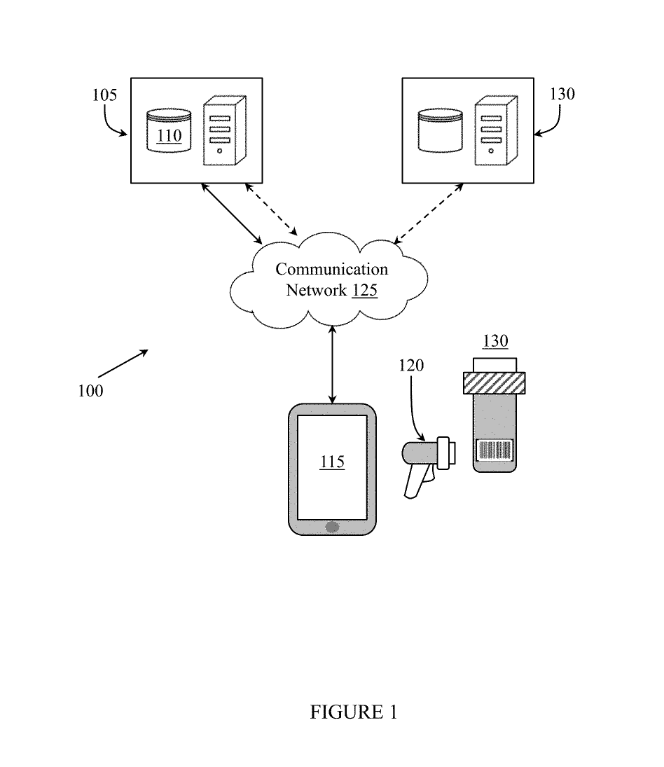

FIG. 1 illustrates an exemplary representation of a sorting assistance system for assisting in sorting of pharmaceutical wastes in accordance with an embodiment of the present disclosure. As shown, the sorting assistance system 100 (hereafter referred as system 100) comprises a server 105 in communication with at least a database 110, an assistance device 115, and a scanner 120, wherein the assistance device 115 and the server 105 are communicatively connected through a communication network 125. In a preferred embodiment, the database 110 is a part of the server 105 and stores composition data of a plurality of pharmaceuticals.

It is to be noted that the most of the dispensaries and pharmacies (medical facilities) often buy the pharmaceuticals in bulk, and create a sub categorization and barcodes that are specific to the medical facilities. Hence, in one embodiment of the present disclosure, the database 110 is created for a specific hospital or pharmacy depending on their formularies. Further, the system 100 may communicate with one or more third party databases (for example, server with database 130) storing composition data of a plurality of pharmaceuticals. It is to be noted that the system 100 may comprises one or more assistance devices located at various medical facilities for assisting users in sorting of pharmaceutical wastes generated at the medical facilities. However, only one assistance device is shown in FIG. 1 for ease of illustration and understanding.

The server 105 may include, for example, a computer server or a network of computers or a virtual server which provides functionalities or services for other programs or devices such as for the assistance device 115. In one implementation, the server 105 is a cloud server comprising one or more processors, associated processing modules, interfaces and storage devices communicatively interconnected to one another through one or more communication means for communicating information. The storage devices within the server 105 may include volatile and non-volatile memory devices for storing information and instructions to be executed by the one or more processors and for storing temporary variables or other intermediate information during processing. In one embodiment of the present disclosure, the server 105 comprises a database 110, the database 110 storing composition data of the plurality of pharmaceuticals. That is, the database 110 stores plurality of pharmaceutical names and associated composition data in a structured format, wherein the composition data may include details of active and inactive substances, chemical properties of the pharmaceutical, etc. Typically, such composition data uniquely identifies the category of the pharmaceuticals as hazardous, non-hazardous, hazardous-P/U (P-listed and U-listed substances), flammable or toxic, for example. In an alternative embodiment, the server 105 is communicatively connected with one or more third party databases (for example, 130 associated with a manufacturer) storing the composition data of the plurality of pharmaceuticals.

Further, the server 105 maintains a local database storing information pertaining to each of the assistance device, wherein the information comprises assistance device ID, location of the assistance device, end user details associated with the assistance device, etc. Furthermore, the server 105 is configured to store pharmaceutical wastage data uploaded by each of the assistance device in a structured manner in the local database for pharmaceutical usage tracking and analysis. For example, pharmaceutical wastage data pertaining to a “Hospital A” is maintained separately so as to enable tracking and further analysis by the operators. Similarly, the pharmaceutical wastage data pertaining to a group of hospitals (hospital chain) is maintained in a structured manner so as to enable tracking and analysis by the operators or authorized persons.

The assistance device 115 may be one of a smartphone, a personal data assistant (PDA) or any general purpose computer capable of connecting to the internet and having other communication capabilities. The assistance device 115 may communicate with the server 105 through the communication network 125 in one or more ways such as wired, wireless connections or a combination thereof. It will be appreciated by those skilled in the art that the assistance device 115 includes one or more processors for processing data, one or more memory units for instructions and data storage, and other known functional units enabling communication between the assistance device 115 and the server 105 for data exchange. In one implementation, the assistance device 115 is communicatively connected, by means of wired or wireless connection, with the scanner device 120 for reading a machine readable code affixed to a pharmaceutical 130 to be sorted. In another implementation, built in sensors, for example a camera, of the assistance device 115 may be used for reading the machine readable code affixed to the pharmaceutical 130. In yet another implementation, the user may verbally read out the pharmaceutical name or any identifier to the assistance device 115, which would be included by the application.

In one embodiment of the present disclosure, the assistance device 115 is installed with a dedicated application which facilitates sorting of the pharmaceutical wastes. It is to be noted that each assistance device is assigned a unique ID which uniquely identifies each assistance device among a plurality of assistance devices within the system 100. Further, such IDs are mapped and stored with the location, date, time and end user details in the local database associated with the server 105. For example, one or more device IDs associated with the one or more assistance devices used by a “medical facility A” are recorded in the local database associated with the server 105.

The communication network 125 may be a wireless network or a wired network or a combination thereof. Wireless network may include long range wireless radio, wireless personal area network (WPAN), wireless local area network (WLAN), mobile data communications such as 3G, 4G or any other similar technologies. The communication network 125 may be implemented as one of the different types of networks, such as intranet, local area network (LAN), wide area network (WAN), the internet, and the like. The communication network 125 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like. Further the communication network 125 may include a variety of network devices, including routers, bridges, servers, modems, computing devices, storage devices, and the like. In one implementation, the communication network 125 is the internet which enables communication between the assistance device 115 and the sever 105.

As described, the sorting assistance system assists the users in sorting of pharmaceutical wastes into a plurality of bins. The manner in which the system functions is described in detail further below.

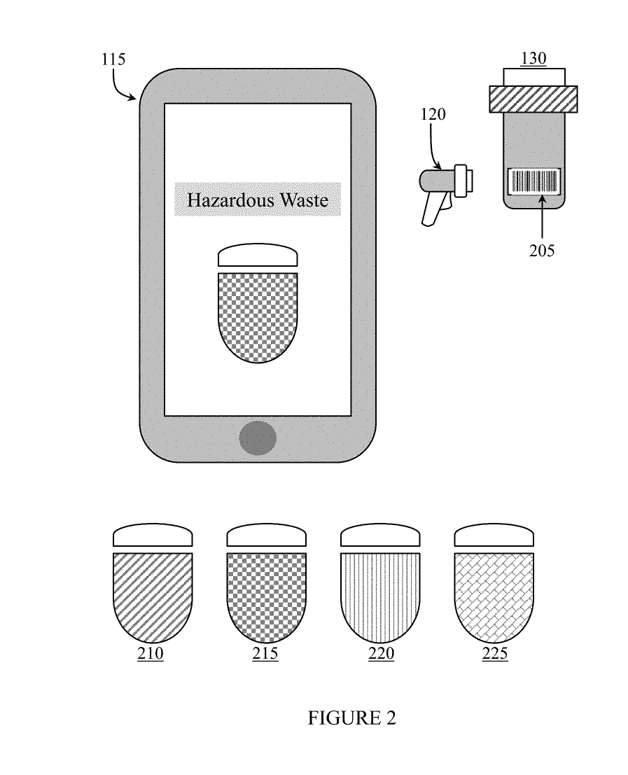

FIG. 2 illustrates a pharmaceutical waste sorting method using sorting assistance system in accordance with an embodiment of the present disclosure. Typically, each pharmaceutical (for example pharmaceutical 130) comprises a machine readable code 205 affixed to it or affixed to its container, as shown, and encodes an identifier (pharmaceutical identifier) which uniquely identifies the pharmaceutical. The pharmaceutical identifier may be a trade name, a generic name, a National Drug Code (NDC) and the like. During sorting, an operator/a user launches the application installed in the assistance device 115, picks up the pharmaceutical to be sorted 130 and scans the machine readable code 205 affixed on the pharmaceutical 130 using the scanner device 120. Alternatively, if the assistance device 115 is scanner enabled, then the user may scan the machine readable code 205 using the assistance device 115, i.e., using an option provided in the application. Upon scanning, the application reads and decodes the machine readable code and identifies the pharmaceutical.

Then, the application fetches the relevant composition data from the at least one database 110. That is, upon identifying the pharmaceutical, the assistance device 115 sends a query to the server 105, the query comprising request for composition data associated with the identified pharmaceutical. The server 105 in turn fetches the relevant composition data from the database 110 or from the third party database and communicates the same to the assistance device 115. As described, the database 110 may be universal database storing composition data of plurality of pharmaceuticals or may be a database created for a specific hospital or pharmacy depending on their formularies. Hence, the server 110 may fetch relevant composition data from any of such databases and communicates the same to the assistance device 115. Upon receiving the composition data, the application determines a category of the pharmaceutical as one of hazardous, non-hazardous, hazardous-P/U (P-listed and U-listed substances), flammable or toxic, for example. In another embodiment, the composition data may also be pre-fetched by the application during low usage times on the assistance device 115 from server 105 or pre-loaded during initial installation of the application. The end user can chose whether to use a local database if the network connection 125 is unavailable or use the connected database 110. That is, a local database comprising composition data of plurality of pharmaceuticals may be maintained on the assistance device 115 and the same may be referred for identifying the composition data associated with the pharmaceutical to be sorted.

In one embodiment of the present disclosure, the application determines the category of the pharmaceutical waste using the composition data and a logic engine, the logic engine defining one or more rules for determining the category. For example, the logic engine is executed at every instance of scanning and sorting, and the rules are pre-programmed rules based on domain knowledge and regulations. For instance, a rule may define that a given pharmaceutical composition having toxicity higher than a pre-defined threshold must be included in category “hazardous”. Further, such rules may be updated remotely using the server 105. Further, a local copy of the rules is also made available as part of the rules engine during the non-availability of communication network 125.

Upon identifying the category, the application may indicate the same (i.e., the type of waste that the user has to dispose) to the user through one or more notification means, for example as a message as shown, a sound, flashing screen, colour changing screen, etc.

In one embodiment of the present disclosure, one or more bins are provided for sorting the pharmaceutical wastes, wherein each bin is assigned for a particular category of pharmaceutical waste and each bin is identified using a visual indicator, for example colour. Referring to FIG. 2, different coloured bins 210 to 225 (indicated by different shades) may be assigned to non-hazardous, hazardous, hazardous-P/U and flammable or toxic categories respectively. Upon determining the category of the pharmaceutical waste to be disposed, the application indicates the particular bin in which the pharmaceutical waste is to be disposed. For example, upon determining that the category of the pharmaceutical waste as “hazardous”, the application displays the visual indicator on the display module of the assistance device 115 as shown, thereby notifying the user to dispose the pharmaceutical waste in the correct bin.

In one embodiment of the present disclosure, the assistance device 115 is further configured for uploading the pharmaceutical wastage data with the local database associated with the server 105 for pharmaceutical usage tracking and analysis, wherein the pharmaceutical wastage data may include, pharmaceutical identifier, the category, and the unique ID of the assistance device 115. That is, upon determining the category of the pharmaceutical waste, the application records the pharmaceutical wastage data in the local database associated with the server 105. This enables the operators to keep track of the categories and compliance and also the consumption of the pharmaceuticals in real time. Further, the operators may use such information for pharmacy scheduling, delivery planning and for adjusting the delivery of new batch of pharmaceuticals to the medical facilities. This can provide optimization of the pharmaceutical inventory at hospitals, reducing inventory carrying costs and pilferage.

It is to be noted that the application and the database is also password protected to avoid data corruption erroneously or purposely by a user or an operator. The security of the assistance device is further enhanced by splitting the databases between the primary storage of the assistance device 115 and a removable storage device (often called an SD Card, or a USB flash drive, a CD-ROM). The primary application and the rules engine are maintained on the primary storage of the assistance device 115 and the database is maintained on the removable device. This makes the entire application secure and unable to be used upon removal of the removable storage card. Since most locations are under secure and locked areas of the hospital, it is very important to enable the staff with an official copy of the removable device.

The system and method disclosed in the present disclosure, provides an efficient solution for managing and sorting pharmaceutical wastes in various medical facilities. Depending on the requirements of the medical facility, the system may be implemented for a specific medical facility (for example for a hospital or a hospital chain) by building a specific custom database depending on their formularies. This provides efficient solution for managing and sorting pharmaceutical wastes in various medical facilities.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

We claim:

1. A sorting assistance system for assisting in sorting pharmaceutical wastes into a plurality of bins, the system comprising:
a server in communication with at least one database storing composition data of a plurality of pharmaceuticals; and
an assistance device in communication with a scanner device and the server, wherein the assistance device is configured for:
reading a machine readable code affixed to a pharmaceutical to be sorted using the scanner device, the machine readable code comprising a pharmaceutical identifier;
identifying the pharmaceutical upon reading the machine readable code;
receiving the composition data associated with the identified pharmaceutical from the at least one database through the server;
determining a category of the pharmaceutical using the composition data and a logic engine, the logic engine defining one or more rules for determining the category;
determining a bin out of the plurality of bins for placing the pharmaceutical based on the identified category, wherein each bin corresponds to one of the category and is identified with an unique visual indicator; and
displaying the visual indicator corresponding to the determined bin, on a screen of the assistance device.

2. The sorting assistance system as claimed in claim 1, wherein the assistance device comprises a processor, a memory and a communication means.

3. The sorting assistance system as claimed in claim 1, wherein the assistance device is uniquely identified using a unique identifier.

4. The sorting assistance system as claimed in claim 1, wherein the unique visual indicator comprises a colour, characters, or combination thereof.

5. The sorting assistance system as claimed in claim 1, wherein the at least one database storing composition data of a plurality of pharmaceuticals is one of an universal database storing the composition data of a plurality of pharmaceuticals or a specific database associated with a medical facility or a third party database.

6. The sorting assistance system as claimed in claim 1, wherein the assistance device is further configured for uploading the pharmaceutical wastage data with a local database associated with the server for pharmaceutical usage tracking and analysis, wherein the pharmaceutical wastage data comprises, pharmaceutical identifier, the category, date and time and the unique ID of the assistance device.

7. The sorting assistance system as claimed in claim 1, wherein the at least one database further comprises metadata associated with the plurality of pharmaceuticals.

8. A method for sorting pharmaceutical wastes into a plurality of bins, the method comprising:
reading, by an assistance device, a machine readable code affixed to a pharmaceutical to be sorted using a scanner device, the machine readable code comprising a pharmaceutical identifier;
identifying, by an assistance device, the pharmaceutical upon reading the machine readable code;
receiving, by an assistance device, a composition data associated with the identified pharmaceutical from an at least one database through a server, the database storing composition data of a plurality of pharmaceuticals;
determining, by the assistance device, a category of the pharmaceutical using the composition data and a logic engine, the logic engine defining one or more rules for determining the category;
determining a bin out of the plurality of bins for placing the pharmaceutical based on the identified category, wherein each bin corresponds to one of the category and is identified with an unique visual indicator; and
displaying the visual indicator corresponding to the determined bin, on a screen of the assistance device.

9. The method as claimed in claim 8, wherein the method further comprises, uploading the pharmaceutical wastage data with a local database associated with the server for pharmaceutical usage tracking and analysis, wherein the pharmaceutical wastage data comprises, pharmaceutical identifier, the category, date and time and the unique ID of the assistance device.
